(19) United States
(12) Patent Application Publication
(10) Pub. No.: US 2015/0374637 A1
(43) Pub. Date: Dec. 31, 2015

(54) SEMI-PERMEABLE CAPSULAR MEMBRANE WITH TAPERED CONDUITS FOR DIABETES FUNCTIONAL CURE

(71) Applicant: Taylor Gun-Jin Wang, Los Angeles, CA (US)

(72) Inventor: Taylor Gun-Jin Wang, Los Angeles, CA (US)

(21) Appl. No.: 14/596,133

(22) Filed: Jan. 13, 2015

Related U.S. Application Data
(60) Provisional application No. 62/018,472, filed on Jun. 27, 2014.

(51) Int. Cl. A61K 9/50; A61K 9/70; A61K 35/39

(52) U.S. Cl. CPC A61K 9/5073; A61K 35/39; A61K 9/5036; A61K 9/5042; A61K 9/5031; A61K 9/7007

(57) ABSTRACT

Some embodiments of the present disclosure include an encapsulated islet for treating diabetes. The encapsulated islet may include a semi-permeable capsular membrane having a plurality of layers including an outer immunoprotection layer, a bridging layer, and an inner backbone layer, each layer having a plurality of pores, wherein the pores increase in size from the immunoprotection layer to the backbone layer, creating the tapered conduits. The semi-permeable capsular membrane may include the following layers, in order from outermost layer to innermost layer: an immunoprotection layer, a bridging layer, and a backbone layer. With proper balancing of membrane thickness and tapered pore size distribution, the encapsulated islets may offer a diabetes treatment or functional cure without immunosuppressive drugs.

### RELATED APPLICATION

This application claims priority to provisional patent application U.S. Ser. No. 62/018,472 filed on Jun. 27, 2014, the entire contents of which is herein incorporated by reference.

### BACKGROUND

The embodiments described herein relate generally to treatments for endocrine disorders, such as diabetes or hypothyroidism, a neurological disorder, or any other disorder able to be treated with cell therapy, and more particularly, to encapsulated pancreatic islets comprising semi-permeable capsular membrane with tapered conduits.

Diabetes is a difficult disease to manage and treat. Conventionally, there are two acceptable treatment protocols for insulin-dependent diabetes mellitus (IDDDM): pancreases/pancreatic islet transplantation and insulin injection or the use of an insulin pump. Pancreases/pancreatic islet transplantation provides good management of diabetes, but its adoption has been limited by the side effects of immunosuppressive drugs. Insulin injection or use of an insulin pump is less invasive and requires no immunosuppressive drugs, but, for many patients, blood glucose control is inadequate. Neither treatment is satisfactory.

Encapsulated pancreatic islets (a bioengineering project) has long been considered as one of the most promising alternative treatment protocols for diabetes, wherein a thin semi-permeable islet encapsulation membrane was assumed to have “uniform pores” that could protect cells from immune attack and, at the same time, allow the influx of molecules important for cell function/survival and efflux of the other desired cellular products. The thin membrane model utilized modifications in the procedure originated by Lim and Sun. It has worked well in small animals. However, the thin membrane model was less than satisfactory in large animal trials. The “uniform pores” assumption was flawed. Capsular membranes for islet transplantation were polymers. Polymeric membranes by nature were random network with non-uniform pore sizes. Size exclusion chromatography measurement has shown thin membranes with pore size distribution cutoffs about 15 nm in diameter include enough large pores for immune system IgG (˜19 nm) to go through. The thin membrane model could not provide adequate immunoprotection.

To address this limitation, a thick membrane model was tested in canine transplantation. The thick membrane with a pore size distribution cutoff of about 15 nm in diameter would allow small particles, such as nutrients and oxygen to enter the membrane with ease. However, large immune system (IgG˜19 nm) would be stopped or snared by those 15 nm pores along the way. This is an accumulative effect—the thicker the membrane, the more efficient the immunoprotection. With proper selection of membrane thickness and pore size distribution, encapsulated canine islets has normalized fasting blood glucose levels in nine out of nine dogs for up to two hundred and fourteen days with a single transplantation. No immunosuppression or anti-inflammatory therapy was used or necessary. However, upon closer examination, the thick membrane model insulin release was found to be wanting. When challenged, the fasting circulating blood glucose level rose much higher than normal and took much longer than normal to return to its baseline. For a membrane with a pore size distribution cutoff of about 15 nm in diameter, there were about 5% of pores smaller than insulin (about 4 nm in diameter). Those small pores would delay or stop insulin from leaving. Like immunoprotection, this is an accumulative effect—the thicker the membrane, the longer the delay. These delays hastened the return of diabetes in less than ⅗ of a year.

If encapsulated islet transplantation is to be offered as a viable option for diabetic management in humans, encapsulated islet transplantation must be able to keep the patient healthy and encapsulated islets functioning for years, not just for months. Transplantations of encapsulated islets must be able to restore patient's health, and not just provide a short reprieve. None of the current capsular designs could meet this challenge. This was likely to be one of the reasons why the encapsulation system has been a “could be” for the diabetes management.

What is needed is a new capsular membrane design that can offer islet immunoprotection of a thick membrane, and insulin release of a thin membrane.

### SUMMARY

Some embodiments of the present disclosure include an encapsulated islet for treating diabetes. The encapsulated islet may include a semi-permeable capsular membrane having tapered conduits and a plurality of layers including an outer immunoprotection and an inner backbone layer, each layer having a plurality of pores, wherein the pores increase in size from the immunoprotection layer to the backbone layer, creating the tapered conduits. These layers are made out of similar polymer compositions of different concentrations that cross-linked well to form a stable membrane. The semi-permeable capsular membrane may include the following layers, in order from outermost layer to innermost layer: an immunoprotection layer, a bridging layer, and a backbone layer. Each of these layers has a plurality of pores; wherein the pores increase in size from the immunoprotection layer to the backbone layer, by staking those layers accordingly, pores of those layers forms tapered conduits. Tapered geometry offers large pores size distribution at interior section of membrane for better insulin release, and small pores size distribution at outer layer for improved immunoprotection. Gradual changing pore size distribution offers better impedance matching for smooth transition. By adjusting the membrane thickness and tapered pore size distribution, insulin release may be increased without compromising the immunoprotection.

### BRIEF DESCRIPTION OF THE FIGURES

The detailed description of some embodiments of the invention is made below with reference to the accompanying figures, wherein like numerals represent corresponding parts of the figures.

FIG. 1 is a perspective view of one embodiment of the present invention.

FIG. 2 is a cutaway/detail perspective view of one embodiment of the present invention.

FIG. 3 is a section detail view of one embodiment of the present invention.

FIG. 4 is a section view of one embodiment of the present invention shown in use.

FIG. 5 is a graphical result of NHP 4510 transplantation experiment.

FIG. 6 is a graphical result of NHP 3912 transplantation experiment.

FIG. 7 is a graphical result of NHP 3912 transplantation experiment.

### DETAILED DESCRIPTION OF CERTAIN EMBODIMENTS

In the following detailed description of the invention, numerous details, examples, and embodiments of the invention are described. However, it will be clear and apparent to one skilled in the art that the invention is not limited to the embodiments set forth and that the invention can be adapted for any of several applications.

The device of the present disclosure may be used to treat diabetes, allowing the insulin to be sufficiently released into the body while also sufficiently blocking the immune response and may comprise the following elements. This list of possible constituent elements is intended to be exemplary only, and it is not intended that this list be used to limit the encapsulated islets of the present application to just these elements. Persons having ordinary skill in the art relevant to the present disclosure may understand there to be equivalent elements that may be substituted within the present disclosure without changing the essential function or operation of the device.
1. Encapsulated Islet
2. Semi-Permeable Capsular Membrane with Tapered Conduits

The various elements of the encapsulated islet of the present disclosure may be related in the following exemplary fashion. It is not intended to limit the scope or nature of the relationships between the various elements and the following examples are presented as illustrative examples only.

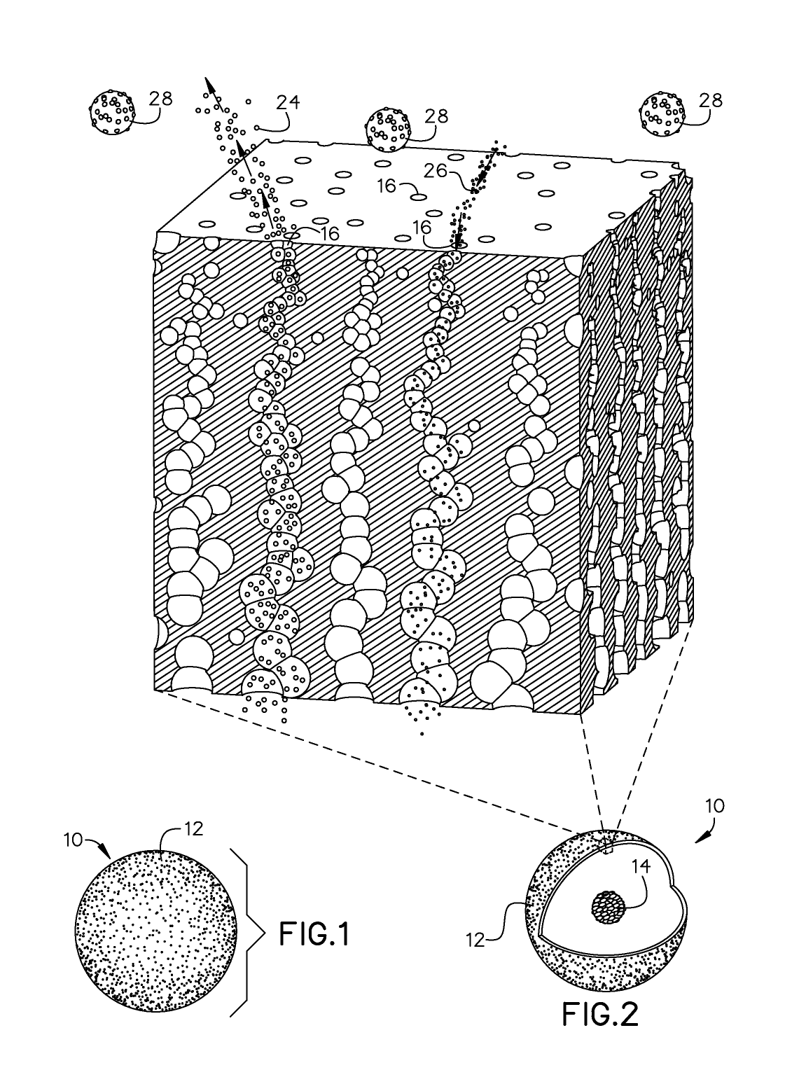

By way of example, and referring to FIGS. 1-4, some embodiments of the present invention comprise an encapsulated islet 10 comprising a semi-permeable capsular membrane 12 having tapered conduits 22, wherein the conduits 22 are configured to sufficiently release insulin into the body while simultaneously blocking the natural immune response. For example, the semi-capsular membrane 12 may comprise a plurality of layers, wherein each membrane, from an immunoprotection layer 34 to a bridging layer 32 to a backbone layer 30, comprises increasingly larger pores, resulting in an overall structure having tapered conduits 22. Specifically, by adjusting the concentrations of polymer constituents and reaction times, layers with different port sizes may be formed. By setting up the layers up in the right order, a membrane with tapered conduits may be formed.

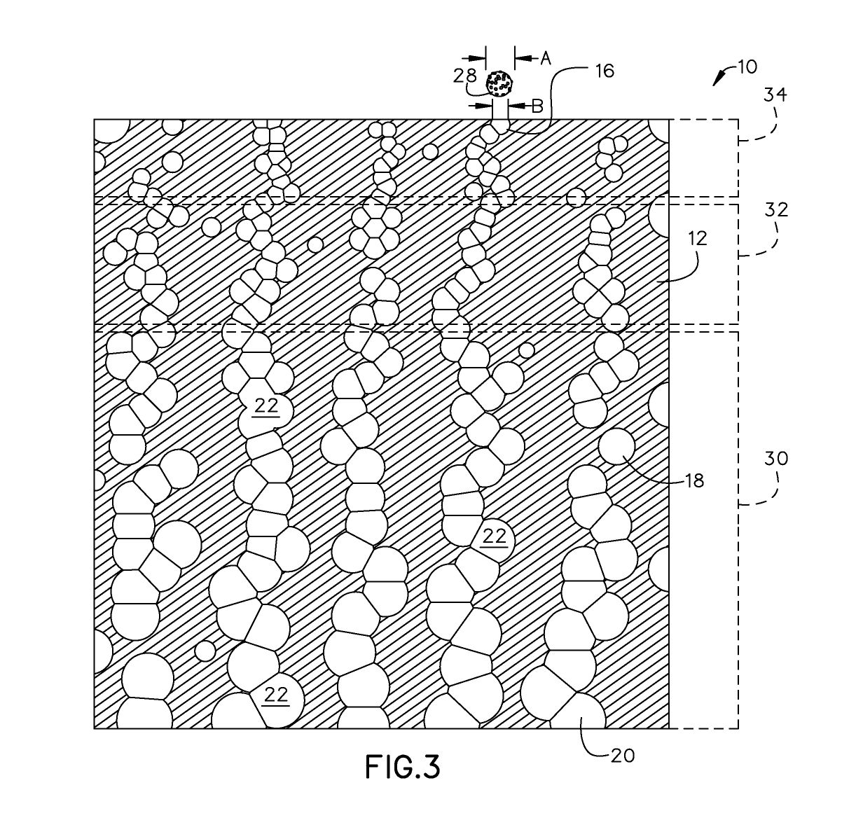

As shown in FIG. 3, some embodiments of the semi-permeable capsular membrane 12 may comprise three layers, wherein the outermost layer (Layer 3) comprises an immunoprotection layer 34, the central layer (Layer 2) comprises a bridging layer 32, and the innermost layer (Layer 1) comprises a backbone layer 30, wherein the layers comprise pores that increase in size from an outermost layer to an innermost layer, allowing increased insulin release while simultaneously providing protection against immune systems. In embodiments, each layer may comprise different concentrations of similar polymer compositions for different pore size distributions. Reaction time may also be used to fine-tune the pore size distribution. The layers with similar polymer compositions may be cross-linked to form a stable, semi-permeable capsular membrane 12. In embodiments, the semi-permeable capsular membrane 12 may further comprise an outermost ablation layer.

The ablation layer may protect the islets from the post-transplant immune surge by shedding membrane material continuously and may comprise a polymer comprising CaCl₂ and SA. The ablation layer may have pores with a diameter of about 60 nm or smaller.

The immunoprotection layer 34 may comprise a thin polymer membrane comprising polymethylene-co-guanidine (PMCG)-cellulose sulfate (CS)/poly L-lysine (PLL), and sodium alginate (SA). Thus, the immunoprotection layer 34 may comprise a polymer membrane of PMCG-CS/PLL-SA. The immunoprotection layer 34 may comprise a plurality of immunoprotection pores 16 with a pore size distribution cutoff about 15 nm in diameter and about 1 μm in thickness.

The bridging layer 32 may comprise a membrane comprising PMCG-CS/PLL-SA-calcium chloride (CaCl₂). Thus, the bridging layer 32 may comprise a polymer membrane of PMCG-CS/PLL-SA-CaCl₂. The bridging layer 32 may comprise a plurality of bridging pores 18 having a pore size distribution cutoff gradually decreasing from about 20 nm to about 15 nm in diameter and less than or equal to about 2 μm in thickness. The bridging layer 32 may be configured to ease the transition and improve the mass transport between the outer, immunoprotection layer 34 and the inner, backbone layer 30.

The backbone layer 30 may be the innermost layer and may comprise a polymer membrane comprising PMCG, CS/CaCl₂, and SA. Thus, the backbone layer 30 may comprise a polymer membrane of PMCG-CS/SA-CaCl₂. The backbone layer 30 may comprise a plurality of backbone pores 20 having pore size distribution cutoffs gradually decreasing from about 30 nm to about 20 nm in diameter and about 8 μm in thickness.

In one particular embodiment, the backbone layer 30 may comprise backbone pores 20 with a pore size distribution cutoff of about 36 nm in diameter, the bridging layer 32 may comprise bridging pores 18 with a pore size distribution cutoff of about 20 nm in diameter, and the immunoisolation or immunoprotection layer 34 may comprise immunoprotection pores 16 with a pore size distribution cutoff of about 15.6 nm in diameter.

Interconnecting these pores of the different layers together formed the tapered conduits 22. The tapered geometry of the conduits 22 results in increased insulin release with the larger inlet at the inner surface, while simultaneously maintaining good immunoprotection with small pores at the exterior surface, as shown in FIGS. 2 and 3. The glucose inflow and insulin outflow may be explicitly linked, wherein they are two legs of a double concentration gradient diffusion driven convection. Convective flow with enlarged conduit diameters improves the mass transport on both flow directions. Thus, both glucose uptake and insulin release are increased. Together, they improve encapsulated islet performance and diabetic management.

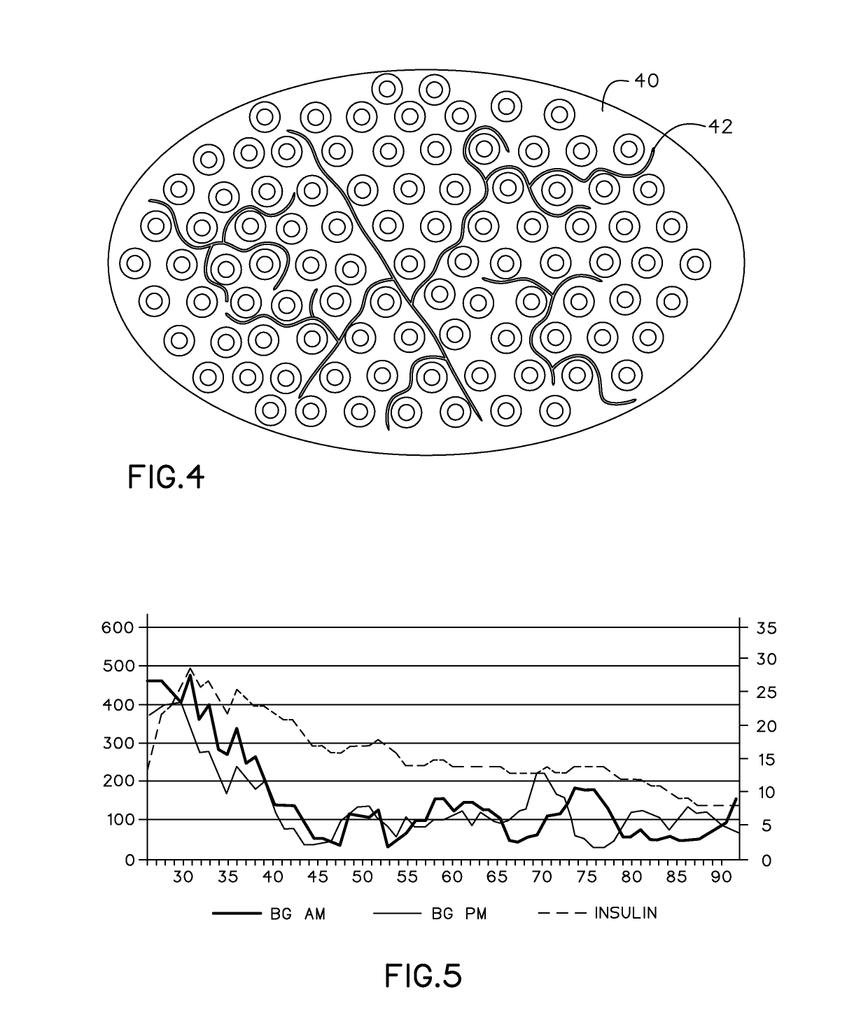

Because humans and non-human primates (NHP) both have erect posture and bipedal locomotion, gravitational forces may push transplanted capsules to fall to the bottom of the pelvis or subcutaneous pockets, forming clumps. Thus, a capsule-patch may be used in conjunction with the capsules to keep them in place and withstand a patient's physical movement and gravitation forces. The patches may be surgically placed at the desired intraperitoneal or subcutaneous site of the primate. The capsule patch may be lightweight and have a large surface/capsule ratio to keep the patch in place. The flexibility of the patches may also allow them to conform to the contour of transplantation sites to promote neovascularization on the dorsal surface of the embedded encapsulated islets, as shown in FIG. 4. The neovascularization may allow oxygen, nutrients, and hormones to be transported to and from the capsule patch with greater efficiency. When the capsules are uniformly spaced, the possibility of islets' hypoxia from being too close to each other may be reduced.

### Immunoprotection Study

The objective of this NHP xeno-transplantation experiment (human donors) was to study the immunoprotection efficacy of the tapered conduit encapsulation system. NHP 4510 (5 kg in body weight BW) received 6 capsule patches containing a total of about 1,350,000 human islets, which exceeded 9 times of islet packing density needed for human allotransplantation. About 3 weeks after the incubation period, the exogenous insulin requirement for NHP 4510 started to drop. It gradually fell from 25-30 unites/day to 7-10 units/day in 90 days with good glycemic control, as shown in FIG. 5. This suggested the tapered conduit capsule design was able to provide good immunoprotection in an extremely oxygen and nutrient challenged xenotransplantation environment with no immunosuppressive or anti-inflammatory drugs.

### Mass Transport Study

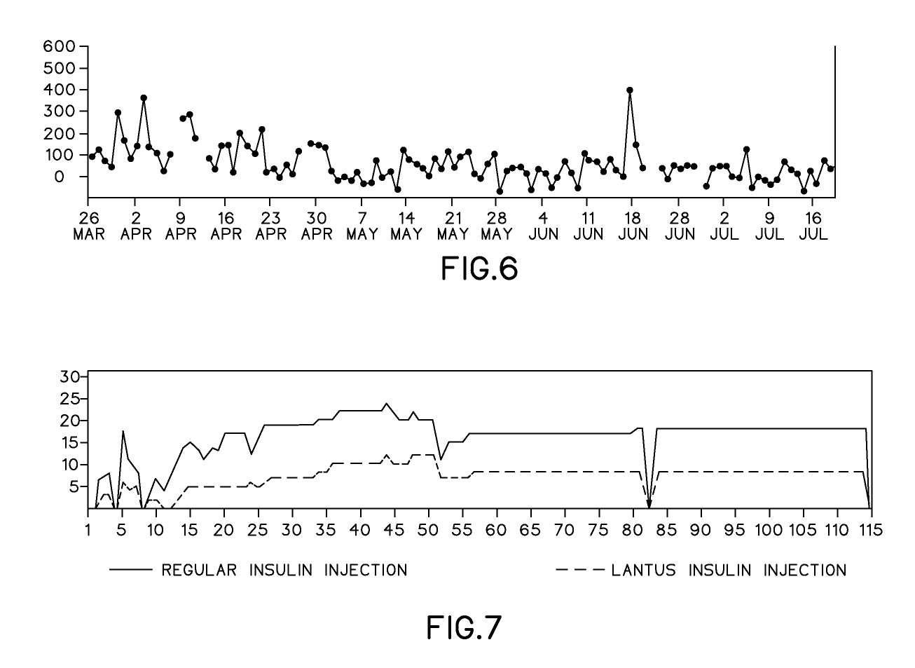

The objective of this NHP allo-transplantation experiment was to study the diabetes management performance of the tapered conduit encapsulation system. NHP 3912 (5 kg in BW) received 12 capsule patches comprising a total of about 180,000 NHP islets (⅙ of a NHP allotransplantation). Supplemental Lantus and regular insulin were provided for basal and meal requirements. Encapsulated NHP islets were to provide self-regulated dosage of corrective insulin for diabetic management. These capsules were transplanted on subcutaneous fat tissue, as shown in FIG. 4. After a 3-week incubation period, encapsulated islets were able to maintain good glycemic control by keeping BG fluctuations within acceptable ranges, as shown in FIGS. 6 and 7. Four-months post-transplantation, the encapsulated islets were explanted to assess their vitality. The return of diabetes with recurrent hyper and hypo episodes soon after explantation confirmed the contribution of encapsulated islets on diabetic management improvement.

Insulin release data provided additional insight on how the new tapered conduit encapsulation system was functioning. The tapered conduit capsules with 180,000 IEQ encapsulated islets secreted about 6 units of insulin. In comparison, a thick membrane model with random pore size distribution secreted about 12 units of insulin with 710,000 IEQ encapsulated islets. Thus, the tapered conduit capsular design increased insulin output by a factor of 2.

In post islet transplantation, most diabetic patients experienced progressive loss of islet function; eventually insulin injection or islet re-transplantation was needed. In NHP 3912, the animal has shown steady diabetic improvement after the incubation period, as shown in Table 1 below:

| | Days 0-32 | Days 33-65 | Days 66-98 | Days 99-119 |
|---|---|---|---|---|
| Plasma Glucose | 220 ± 74 | 150 ± 56 | 127 ± 42 | 102 ± 37 |
| HbA1c | 9.3 | 6.85 | 6.05 | 5.2 |

The table suggests tapered conduit capsules complement with capsule patch could provide improvements on islet health and functional longevity. Thus, encapsulated islet transplantation may be able to offer type I diabetic patients a functional cure without immunosuppressive drugs.

Use of the encapsulated islets of the present invention, wherein the islet comprise a semi-permeable membrane comprising tapered conduits may treat diabetes. Particularly, use of the tapered conduits may improve insulin transport without adversely impacting immunoprotection.

With respect to type I diabetes, the tapered conduit encapsulation system may offer at least two possible protocols: (1) subtherapeutic dosage transplantation of encapsulated islets may provide self-regulated corrective insulin for diabetic management, which may free patients from constantly worrying about blood glucose and may offer patients lifetime diabetic management with multiple re-transplantations; and (2) therapeutic dosage of tapered conduit system may be able to offer type I diabetic patients an insulin-independent functional cure.

With respect to type II diabetes, the patients may benefit from sub-therapeutic encapsulated islet transplantation in greater numbers by replacing damages islets and helping keep diabetes under control, which may arrest the progression of diabetes before it starts to ravage the patient's body and rob them of their quality of life.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. An encapsulated islet for treating diabetes, the encapsulated islet comprising:
a semi-permeable capsular membrane comprising tapered conduits, wherein:
the semi-permeable capsular membrane comprises a plurality of layers including an outer immunoprotection layer, a bridging layer, and an inner backbone layer, each layer comprising a plurality of pores; and
the pores increase in size from the immunoprotection layer to the backbone layer, creating the tapered conduits, wherein the layers are made of similar polymer compositions configured to cross-link with one another to form a stable membrane.

2. The encapsulated islet of claim 1, wherein the semi-permeable capsular membrane comprises the following layers:
an immunoprotection layer;
a bridging layer; and
a backbone layer, wherein the immunoprotection layer is the outermost layer and the backbone layer is the innermost layer.

3. The encapsulated islet of claim 2, further comprising an outermost ablation layer, such that an order of the layers is the ablation layer, the immunoprotection layer, the bridging layer, and the backbone layer from outermost to innermost layer.

4. The encapsulated islet of claim 3, wherein the ablation layer comprises a polymer comprising CaCl₂ and SA.

5. The encapsulated islet of claim 2, wherein:
the immunoprotection layer comprises a polymer membrane comprising polymethylene-co-guanidine (PMCG)-cellulose sulfate (CS)/poly L-lysine (PLL), and sodium alginate (SA).

6. The encapsulated islet of claim 2, wherein:
the bridging layer comprises a polymer membrane of PMCG-CS/PLL-SA-CaCl₂.

7. The encapsulated islet of claim 2, wherein:
the backbone layer comprises a polymer membrane of PMCG-CS/SA-CaCl₂.

8. The encapsulated islet of claim 2, wherein:
the immunoprotection layer comprises a plurality of immunoprotection pores having a pore size distribution cutoff of about 15 nm in diameter and a thickness of less than or equal to about 1 μm;
the bridging layer comprises a plurality of bridging pores having a size distribution cutoff gradually decreasing from about 20 nm to about 15 nm in diameter and a thickness of less than or equal to about 2 μm; and
the backbone layer comprises a plurality of backbone pores having a size distribution cutoff gradually decreasing from about 30 nm to about 20 nm in diameter and a thickness of larger than about 8 μm.

9. A system for treating diabetes, the system comprising:
a capsule patch configured to be surgically placed at a desired intraperitoneal or subcutaneous site of a diabetic patient; and
a plurality of encapsulated islets held in place by the capsule patch, each encapsulated islet comprising:
a semi-permeable capsular membrane comprising tapered conduits, wherein:
the semi-permeable capsular membrane comprises a plurality of layers including an outer immunoprotection layer, a bridging layer, and an inner backbone layer, each layer comprising a plurality of pores; and
the pores increase in size from the immunoprotection layer to the backbone layer, creating the tapered conduits.

10. The system of claim 9, wherein the semi-permeable capsular membrane comprises the following layers:
an immunoprotection layer comprising a polymer membrane comprising polymethylene-co-guanidine (PMCG)-cellulose sulfate (CS)/poly L-lysine (PLL), and sodium alginate (SA);
a bridging layer cross-linked to the immunoprotection layer, the bridging layer comprising a polymer membrane of PMCG-CS/PLL-SA-CaCl₂,
a backbone layer cross-linked to the bridging layer, the backbone layer comprising a polymer membrane of PMCG-CS/SA-CaCl₂,
wherein:
the immunoprotection layer is the outermost layer and the backbone layer is the innermost layer.
